(19) United States
(12) Patent Application Publication
(10) Pub. No.: US 2025/0250646 A1
(43) Pub. Date: Aug. 7, 2025

(54) DUAL GENETIC REPORTERS IN HUMAN PLURIPOTENT CELLS FOR USE AS ALTERNATIVE TO MOUSE EMBRYO ASSAY

(71) Applicant: Nexpring US Opco Inc., Beverly, MA (US)

(72) Inventors: CHRISTINA TENENHAUS DANN, BLOOMINGTON, IN (US); BRIANNA MICHELE MCSWAIN, INDIANAPOLIS, IN (US); STEVE MULLEN, GREENWOOD, IN (US); KASSI SHELTON, BEDFORD, IN (US)

(21) Appl. No.: 18/856,259

(22) PCT Filed: Apr. 11, 2023
(86) PCT No.: PCT/US2023/065606

§ 371 Date: Oct. 11, 2024

Related U.S. Application Data
(60) Provisional application No. 63/332,782, filed on Apr. 20, 2022.

(51) Int. Cl. C12Q 1/6897; C12N 5/074; G01N 33/50

(52) U.S. Cl. CPC C12Q 1/6897; C12N 5/0696; G01N 33/5014; C12N 2503/02; C12N 2510/00

(57) ABSTRACT

Systems and methods for assessing cytotoxicity of cellular media and devices. Such systems and methods comprising genetically modified stem cells that have been modified to express a reporter upon activation of a gene associated with pluripotency and/or trophectoderm development in the genetically modified stem cells.

### BACKGROUND

The Mouse Embryo Assay (MEA) is the customary bioassay used for assessing the quality of reagents and labware produced in the in vitro fertilization (IVF) industry. In this assay one-cell or two-cell mouse embryos are cultured up until the blastocyst stage in the presence of the test article (e.g. test media or control media exposed to a test device). Blastocyst morphology is evaluated and if at least 80% of the embryos fulfill morphological criteria of a healthy blastocyst, normalized to the percent blastocysts in control conditions, the test article is scored as passing. In addition to being used by manufacturers of IVF media and devices, MEA is also used by individual IVF clinics to fulfill requirements for accreditation in demonstrating embryologist's proficiency in embryo handling and MEA may be used for training entry-level embryologists.

A problem with the MEA is that the outcome is highly dependent on the exact procedure used. For instance, research has shown that starting with one-cell embryos is more sensitive to perturbation than starting at the two-cell stage. A common embryo strain used in MEA's, CBA/B6 hybrid embryos, are less sensitive than other strains, leading to variability in results. Other factors such as the presence or absence in media of proteins such as albumin, which have a “chelating” effect on toxins, also may mask detection of potential contaminants. Furthermore, the MEA outcome is scored based on morphology, which is subjective.

An additional concern with the MEA is that it uses mouse embryos, which may not be relevant models for the sensitivities and requirements of human embryos in culture. Differences between mouse and human embryo development are numerous. For example, the transition from maternal transcription to zygotic transcription in a mouse occurs at the 2-cell stage while in human it is around 4-cell to 16-cell stage. A practical outcome of species differences is that mouse embryos seem to be very robust and often withstand manipulation and perturbation much more readily than human embryos. In addition to the differences between mouse and human embryonic development, there is ethical concern about the large-scale requirement for producing and killing mouse embryos for use in MEAs.

A need remains for materials and assays for assessing the quality of reagents. The present disclosure provides such medical compositions and products, as well as methods for preparing and using the same.

### SUMMARY

In certain aspects, the present disclosure pertains to unique systems and methods for assessing the cytotoxicity of cellular media. In accordance with some forms, such systems and methods comprise one or more genetically modified stem cells. Accordingly, in one form the present disclosure provides a method for assessing cytotoxicity of cellular media and devices, the method comprising exposing a first target media to a first genetically modified stem cell population, wherein the first genetically modified stem cell population comprises stem cells that have been modified to express a first reporter upon activation of a gene associated with trophectoderm development in the genetically modified stem cells, and analyzing the media for expression of the first reporter. In certain embodiments, the gene associated with trophectoderm development is either CGA or CGB. In some forms, the method also comprises exposing a second target media to a second genetically modified stem cell population, wherein the second genetically modified stem cell population comprises stem cells that have been modified to express a second reporter upon activation of a gene associated with pluripotency in the genetically modified stem cells, and analyzing the second target media for expression of the second reporter. In certain embodiments, the gene associated with pluripotency is selected from the group comprising: SOX2, NANOG, and POUF1. In accordance with some forms, the first and/or second genetically modified stem cell population comprises induced pluripotent stem cells.

In another embodiment, the present disclosure provides a system for testing cell culture media, the system comprising a first genetically modified stem cell population, wherein the first genetically modified stem cell population comprises stem cells that have been modified to express a first reporter upon activation of a gene associated with trophectoderm development. In certain embodiments, the gene associated with trophectoderm development is either CGA or CGB. In some forms, the system also comprises a second genetically modified stem cell population, wherein the second genetically modified stem cell population comprises stem cells that have been modified to express a second reporter upon activation of a gene associated with pluripotency in the genetically modified stem cells. In certain embodiments, the gene associated with pluripotency is selected from the group comprising: SOX2, NANOG, and POUF1. In accordance with some forms, the first and/or second genetically modified stem cell population comprises induced pluripotent stem cells.

In another embodiment, the present disclosure provides a cell culture comprising a first genetically modified stem cell population, wherein the first genetically modified stem cell population comprises stem cells that have been modified to express a first reporter upon activation of a gene associated with trophectoderm development. In certain embodiments, the gene associated with trophectoderm development is either CGA or CGB. In some forms, the first reporter is a bioluminescent reporter. In accordance with some forms, the first genetically modified stem cell population comprises induced pluripotent stem cells.

In yet another embodiment, the present disclosure provides a laboratory testing kit comprising a first genetically modified stem cell population, wherein the first genetically modified stem cell population comprises stem cells that have been modified to express a first reporter upon activation of a gene associated with trophectoderm development, wherein said first genetically modified stem cell population is contained within a sterile package. In certain embodiments, the gene associated with trophectoderm development is either CGA or CGB. In some forms, the laboratory testing kit also comprises a second genetically modified stem cell population, wherein the second genetically modified stem cell population comprises stem cells that have been modified to express a second reporter upon activation of a gene associated with pluripotency in the genetically modified stem cells. In certain embodiments, the gene associated with pluripotency is selected from the group comprising: SOX2, NANOG, and POUF1. In accordance with some forms, the first and/or second genetically modified stem cell population comprises induced pluripotent stem cells.

Additional embodiments, as well as features and advantages of embodiments of the invention, will be apparent from the description herein.

### BRIEF DESCRIPTION OF THE DRAWINGS

FIG. 1A depicts the results of three independent tests of SOX2-snLuc cells in the presence of varying concentrations of ethylene glycol for 48 hours. Nanoluciferase quantity normalized to tp 0 h is shown as a fraction of control cells. “TE” refers to a control population of cells exposed to differentiation media instead of pluripotency media.

FIG. 1B depicts the results of three independent tests of CGA-snLuc cells in the presence of BAP differentiation medium with varying concentrations of ethylene glycol for 65 hours. Nanoluciferase quantity and shown as a fraction of control cells. “Pluri” refers to a control population of cells exposed to pluripotency media instead of differentiation media.

FIG. 1C depicts the results of multiple independent tests CGA-snLuc cells in the presence of DAF-BAP differentiation medium with varying concentrations of ethylene glycol for 48 hours.

FIGS. 2A-C depicts immunostaining from side-by-side cultures of CGA-snLuc cells in the presence of DAF-BAP or BAP differentiation medium. Timepoints are represented in days post exposure to each differentiation medium. Images are taken at 10× magnification. FIG. 2A shows the trophectoderm specification transcription factors, GATA 2/3. FIG. 2B shows pan-trophoblast cytoskeletal protein, KRT7. FIG. 2C depicts differentiated syncytiotrophoblast marker, CGA, which is not detectable at the earliest stage of differentiation. Note the presence of cyst-like syncytial structures in DAF-BAP medium.

### DESCRIPTION OF THE SELECTED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated graph, and further applications of the principles of the disclosure as illustrated therein are herein contemplated as would normally occur to one skilled in the art to which the disclosure relates.

As disclosed above, aspects of the present disclosure relate to novel methods and materials for an alternative assay to the MEA using genetically modified stem cells. Thus, the present disclosure provides an assay, materials, and methods of performing an assay for evaluating cytotoxicity of media, in particular media exposed to devices and/or materials used in IVF procedures. The assays of the current disclosure are configured to quantitatively measure key features of early embryonic development. Development of an embryo to the blastocyst stage involves cellular proliferation and formation of the trophectoderm lineage (future placenta) while concurrently maintaining a subset of cells, the inner cell mass, in the pluripotent state. In accordance with certain embodiments, the present disclosure provides for an assay, and materials and methods for performing such assay, comprising at least one genetically modified stem cell line. In certain embodiments the genetically modified stem cell has been modified to secrete a reporter or detectable marker upon reaching a preselected developmental milestone. In accordance with some forms, two or more genetically modified cells lines are used to indicate that the target cells have achieved two or more developmental milestones. Thus, the present disclosure provides an assay capable of validating a target media for its ability to support cells to pluripotency and/or the formation of placental-like cells (trophectoderm). When used in parallel, each in their respective culture conditions, the modified cells provide a read-out for perturbations that would cause the cells to die, reduce proliferation, fail to maintain pluripotency, and/or fail to become differentiated to placenta-like tissue.

In certain embodiments, the present disclosure provides a live-cell reporter for pluripotency. In accordance with certain preferred embodiments, the live-cell reporter comprises a genetically modified stem cell which has been modified with a cell reporter in a gene associated with pluripotency. In certain embodiments, the gene associated with pluripotency comprises SOX2, NANOG, and/or POU5F1 (OCT4).

In certain embodiments, the present disclosure provides a live-cell reporter for trophectoderm differentiation. In accordance with certain preferred embodiments, the live-cell reporter comprises a genetically modified stem cell which has been modified with a cell reporter in a gene associated with trophectoderm development. In certain embodiments, the gene associated with trophectoderm development comprises CGA, CBA, GATA2, GATA3, CDX2, KRT23, KRT7, HAND1, ISL1, TEAD1, TEAD4, TFAP2A, TFAP2C, and/or PGR.

As disclosed herein, the present disclosure provides for a system comprising a stem cell which has been genetically modified to include one or more reporter genes. Any suitable reporter may be used. In certain embodiments the reporter comprises a fluorescent reporter, for example GFP, deGFP, mCherry, Venus, tdTomato and/or mScarlet. In certain embodiments, the reporter comprises a colorimetric reporter, for example lacZ. In certain embodiments the reporter comprises a bioluminescent reporter, for example luxCDABE, Renilla, and/or Nanoluciferase. In certain embodiments, the reporter is secreted into the test media. In these embodiments a portion of the test media may be sampled in order to detect the secreted reporter. In certain embodiments, the reporter is retained within the cell. In some forms, the present disclosure provides for systems and methods utilizing two genetically modified cell lines, one indicating pluripotency and the other indicating trophectoderm development. In accordance with some forms, both modified stem cell lines may be configured to emit the same or different reporters as described above. Additionally, or alternatively, it is within the scope of the disclosure to present a single stem cell line which has been modified to contain two different reporter genes.

In certain embodiments the present disclosure provides for analyzing the target media after it has been exposed to one or more genetically modified stem cells as disclosed herein. Such analysis is performed to detect the presence or lack of the product of the reporter. A person having ordinary skill in the art will readily understand the appropriate analysis needed to determine the presence or lack thereof of the reporter based on the selection of the reporter itself. For example, in certain embodiments such analysis comprises viewing the target media or cells with an ultraviolet or other specialized light source.

In accordance with some forms, the present disclosure provides materials and methods for assessing cytotoxicity of IVF media. The materials and methods of the present disclosure may be used to assess any suitable IVF media capable of supporting embryonic development.

As detailed above the assays disclosed herein utilize one or more genetically modified stem cells. The genetically modified stem cell may comprise any suitable stem cell in which one or more of the genes associated with pluripotency and/or trophectoderm development are expressed. For example, in accordance with certain embodiments the genetically modified stem cell comprises an induced pluripotent stem cell. In some forms the genetically modified stem cell may comprise a mesenchymal stem cell, a trophoblast stem cell, or a placental derived stem cell. It is within the scope of the disclosure to provide an assay including varying stem cell types. For example, the stem cell which has been genetically modified to express a reporter upon activation of a gene associated with trophectoderm development may comprise a different type of cell than the stem cell which has been genetically modified to express a reporter upon activation of a gene associated with pluripotency.

The present disclosure provides systems, kits, and methods for assaying a target media for cytotoxicity. In accordance with some forms, the target media is a cell culture media used for the culture of embryonic cells. In certain embodiments, the target media is a medium which has been exposed to one or more devices. In this way the present disclosure provides methods for assaying cytotoxicity of a media as a means for assaying the cytotoxicity or contamination of a device or material, such as pipets, gloves, or culture dishes, which has contacted the media. In certain embodiments, the systems, kits, methods, and materials of the present disclosure may be used to validate the performance of a technician or process in avoiding contamination of a media.

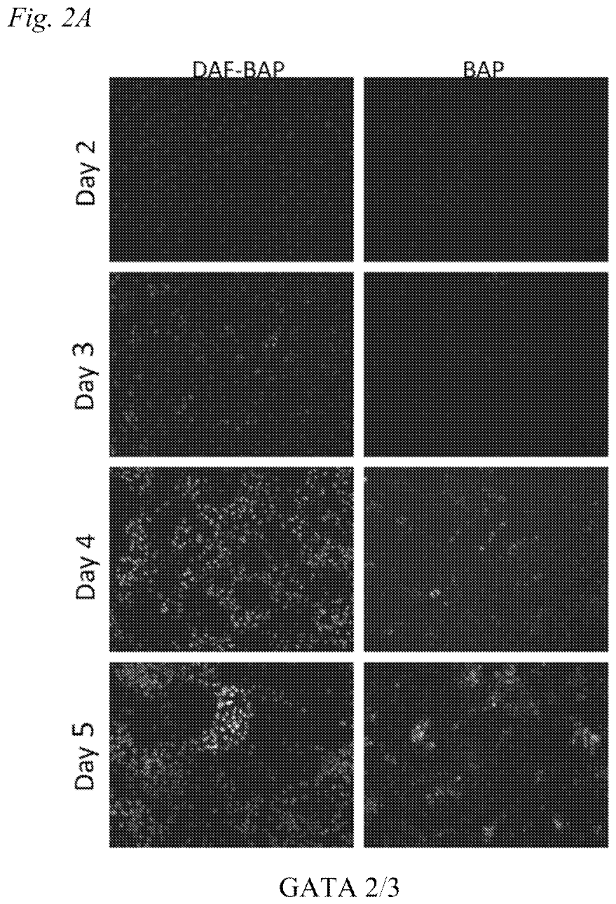

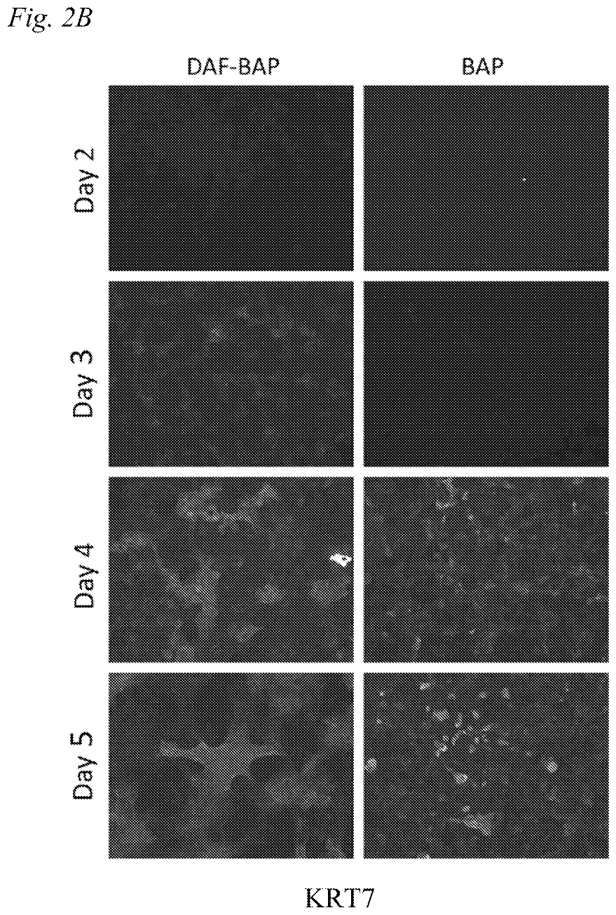

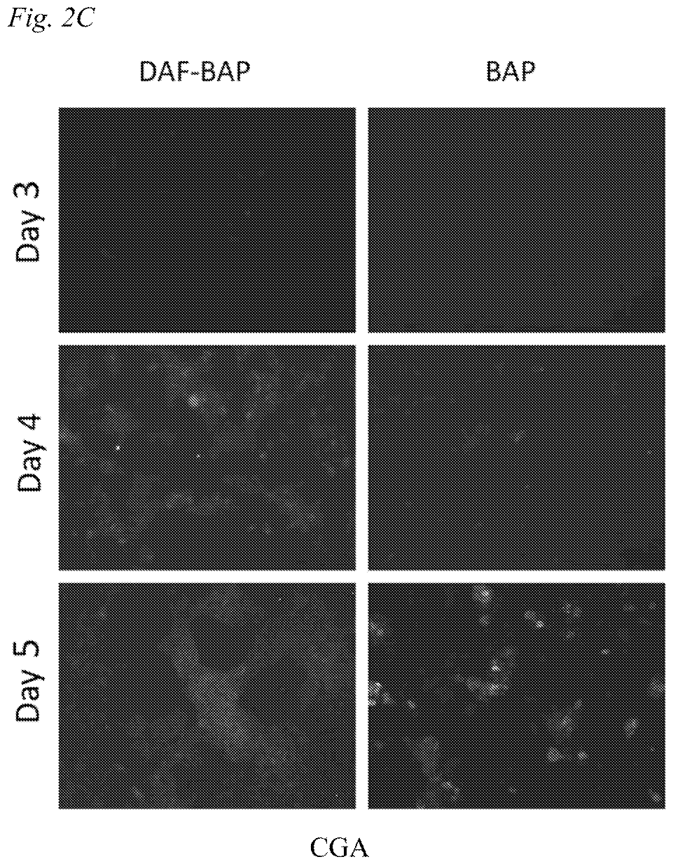

A target media may be exposed to a genetically modified stem cell population for any amount of time sufficient to allow for cellular differentiation and expression of the reporter. In accordance with certain embodiments, for example when validating equipment or techniques, the target media may be selected to support trophectoderm and/or placental differentiation. In accordance with some forms, the target media comprises Essential 8 Flex media. In certain embodiments, genetically modified stem cells as described herein may be cultured in a media containing BMP-4, A83-01, and/or PD173074. In some forms the media comprises a basal media (BAP) comprising BMP-4, A83-01, and/or PD173074. BAP media, as used herein, refers to a basal media configured to stimulate trophectoderm differentiation. In certain embodiments, genetically modified stem cells as described herein may be cultured in a media containing forskolin. For example, in certain embodiments the BAP containing media may include forskolin. Applicants have surprisingly discovered that use of a forskolin containing media accelerates the differentiation of genetically modified stem cells as described herein. FIG. 2 depicts the relative timing of expression of differentiation markers, GATA2/3, KRT7 and CGA and shows the markers becoming detectable about one day sooner during differentiation in DAF-BAP medium than in BAP medium. One exemplary embodiment of such a forskolin containing media, comprises DMEM, Albumin, and Forskolin (DAF-BAP) is shown in table 1 below.

TABLE 1
DAF-BAP media formulation

| | |
|---|---|
| Stock | Per 1 mL |

| | | | |
|---|---|---|---|
| DMEM w/HEPES (Basal) | Basal | 1 | mL |

| | | | | |
|---|---|---|---|---|
| BMP4 | 100 | μg/mL | 1 | μL |
| A83-01 | 10 | mM | 0.1 | μL |
| PD173074 | 1 | mM | 0.1 | μL |

| | | | |
|---|---|---|---|
| Albumin | Powder | 5 | mg |

| | | | | |
|---|---|---|---|---|
| Forskolin | 50 | mM | 0.8 | μL |

Thus, in accordance with certain embodiments, the genetically modified stem cells of the present disclosure may be exposed to the target media for at least 24 hours, preferably at least 36 hours, even more preferably at least 48 hours. In accordance with some forms, the target media is exposed to a genetically modified stem cell which has been modified with a reporter indicating pluripotency, for at least 24 hours, preferably at least 36 hours, even more preferably at least 48 hours. In certain embodiments, the target media is exposed to a genetically modified stem cell for 12 to 120 hours, preferably 18 to 108 hours, even more preferably 24 to 96 hours.

The genetically modified stem cells of the present disclosure may be provided as part of a kit or system. In some forms, genetically modified stem cells are provided frozen and are thawed prior to use. Thus, in accordance with some forms, the present disclosure provides methods of testing a cell culture media comprising thawing a frozen cellular composition comprising genetically modified stem cells as described herein.

The assay may be carried out in various formats. For example, the genetically modified stem cells may be cultured in a traditional 2D adherent format in single or multi-well plates. In another format, embryoid bodies could be formed using the genetically modified stem cells disclosed herein, and assayed while the cells are in 3D structures resembling embryos. In yet another format, frozen stocks of single cells, or embryoid bodies, could be generated and the assay could be performed on thawed cells or embryoid bodies in 3D structures.

Any or all of the components described herein can be provided in a sterile package for providing necessary parts, or a variety of parts, to a user. For example, one or more vials containing genetically modified stem cells may be provided in a single sterile package or kit. Sterilization may be achieved, for example, by irradiation, ethylene oxide gas, or any other suitable sterilization technique, and the materials and other properties of the medical packaging will be selected accordingly. Packages or kits of the components described herein can include additional devices or tools which may be useful in the particular medical procedure being performed.

To promote a further understanding of embodiments disclosed herein and their features and advantages, the following specific Examples are provided. It will be understood that these examples are illustrative and not limiting in nature.

### Example 1

### Generation of Genetically Modified Cell Lines

Live cell reporters for trophectoderm differentiation and pluripotency were created using Crispr genome editing technology. For assessing pluripotency, a transgene was inserted just before the stop codon of the SOX2 gene to be translationally in frame with the SOX2 open reading frame. The transgene consisted of a T2A ribosomal skip sequence, furin cleavage site, and a Nanoluciferase coding sequence, which utilized the IL6 signal peptide. Thus, activation of the genetically modified SOX2 gene would be expected to generate two protein products: intracellular SOX2 and secreted Nanoluciferase. To minimize the possibility of donor DNA or genomic DNA recutting during Crispr-mediated modification, one silent mutation was introduced in the T2A linker region of the donor DNA. Homology arms were approximately 200 nucleotides long.

For assessing trophectoderm differentiation, a live cell reporter for human Chorionic Gonadotropin (hCG) was developed. hCG is a dimer of the protein products of the CGA and CGB genes. A transgene was inserted translationally in frame just after the start codon of the CGA gene. The transgene consisted of the IL6 signal peptide and Nanoluciferase coding sequence without a stop codon, followed by T2A ribosomal skip sequence and furin cleavage site. Thus, activation, of the genetically modified CGA gene would be expected to generate secreted Nanoluciferase and secreted CGA product. Homology arms were approximately 200 nucleotides long.

Transgene inserts were synthesized in the form of plasmid DNA. Double stranded donor DNA fragments were generated by PCR amplification and products were pooled and purified with magnetic beads. Guide RNA sequence for targeting the SOX2 gene was:

5′-TGCCCCTCTCACACATGTGA-3′

and for the CGA gene was:

5′-GTCTGTCTGCAGGAGCGCCA-3′.

Separate cell lines were made for each reporter using commercially available human induced pluripotent stem cells cultured in E8-Flex media on tissue culture plates coated with Vitronectin (rhVTN-N). Ribonucleoprotein particles consisting of 20 pmol guide RNA (gRNA) and 2.5 μg of CAS9 protein were prepared by mixing with Celetrix electroporation buffer and after a brief incubation, mixed with 0.75-1 μg donor DNA and 0.5 million cells. Electroporation was performed using 20 μl Celetrix tubes and following the manufacturer's recommended settings. After a recovery and expansion period, electroporated cells were dissociated with Accutase and plated at very low-density using CloneR supplement to enhance survival, and approximately 50 individual colonies were manually picked to be cultured separately and screened using a combination of PCR and Luciferase detection methods. A second round of low-density plating, colony separation, and screening was performed for each cell line to increase the likelihood of generating clonally derived homogenous cell lines. Sequence analysis was performed on genomic DNA that was amplified in the 5′ junction and 3′ junction regions between transgene and native DNA. Cell lines containing mutations predicted to cause a change to the amino acid sequence were eliminated for further analysis. Each cell line was expanded and cryopreserved to prepare a master cell bank.

### Example 2

### Effect of Ethylene Glycol Adulterant in Differentiation Media

To perform this assay, cells from the cell lines described in Example 1 were thawed using standard procedures and plated on Vitronectin coated wells of a 96-well plate in Essential 8 Flex medium with Revitacell supplement. Plating density for SOX2-snLuc cells was approximately 20,000 cells per well and for the CGA-snLuc cells, approximately 15,000 cells per well. Empty wells were filled with PBS to reduce variation in Nanoluciferase concentration related to evaporation. Cells were allowed to recover for 7-18 hours prior to exposure to test media.

For SOX2-snLuc cells the test media was Essential 8 Flex. For CGA-snLuc cells the activation of the reporter required medium that supported trophectoderm/placental differentiation, such as BAP medium. DAF-BAP medium was an optimized formulation that accelerated differentiation and reporter activation compared to other formulations of BAP medium that lacked forskolin. DAF-BAP contained DMEM with 25 mM HEPES and 4.5 g/L Glucose and without L-Glutamine, 100 ng/ml BMP-4, 1 μM A83-01, 0.1 μM PD173074, 5 mg/mL of AlbumiNZ Bovine Albumin Low Free Fatty Acid, and 40 μM forskolin. Media was pre-warmed prior to forskolin addition to prevent precipitation. Compounds to be tested for toxicity (e.g. ethylene glycol or triton-x-100), or devices and disposables to be tested were mixed with or exposed to test media prior to cell exposure.

Following recovery from thawing, media was exchanged to the respective test medium for each cell line. In the case of CGA-snLuc cells, two partial exchanges amounting to ˜95% total exchange were completed to prevent loss of cell adherence during medium exchange, while still ensuring substantial removal of the pluripotency medium. In the case of SOX2-snLuc cells, a baseline read of Luciferase expression was optionally performed prior to media exchange to assess variations in plating density or post-thaw recovery. Exposure to test medium was for 24-96 hours for SOX2-snLuc cells and 48-96 hours for CGA-snLuc cells. Secreted Nanoluciferase was detected by removing 10% of the culture media and mixing in a white assay plate with substrate and buffer using the Promega Nano-Glo Luciferase Assay kit. Luminescence was measured with a BioTek Cytation 1 imaging reader.

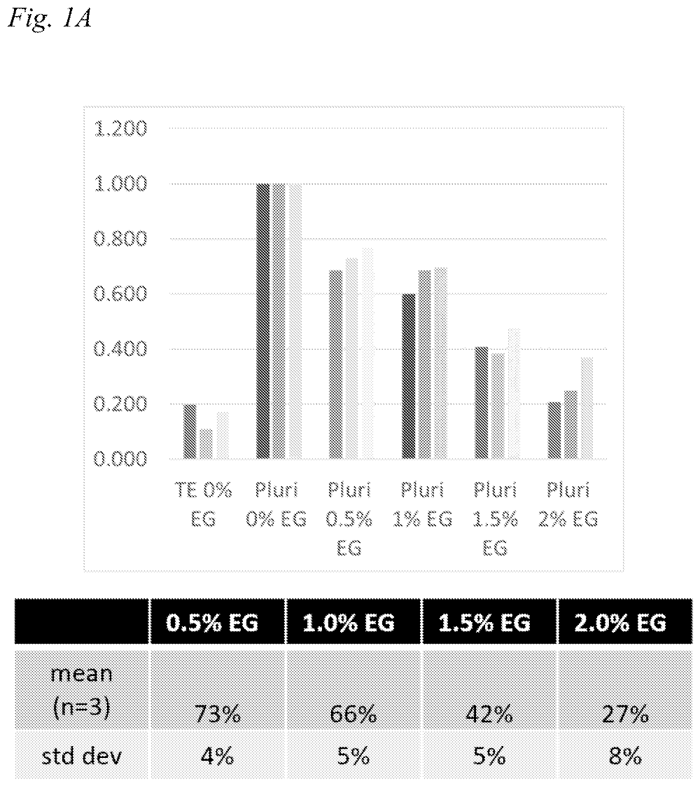

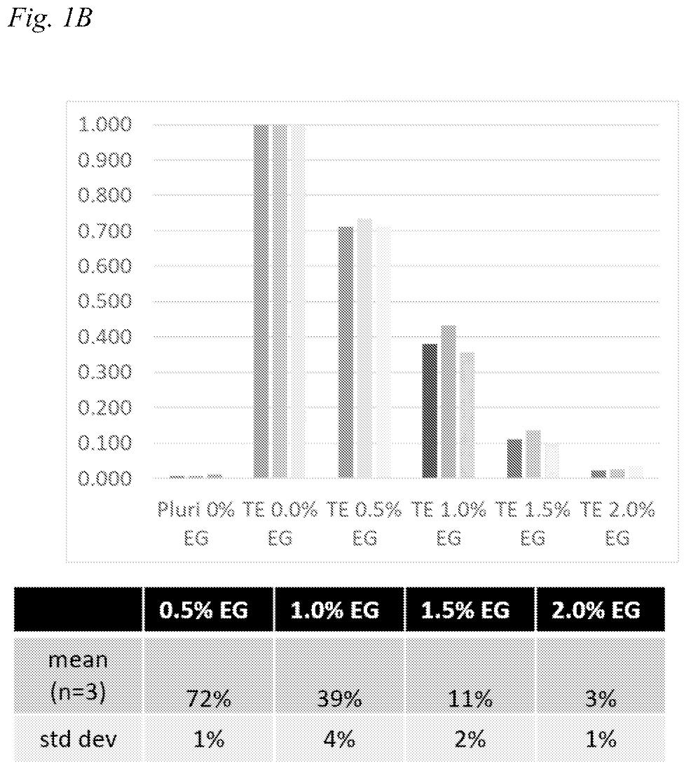

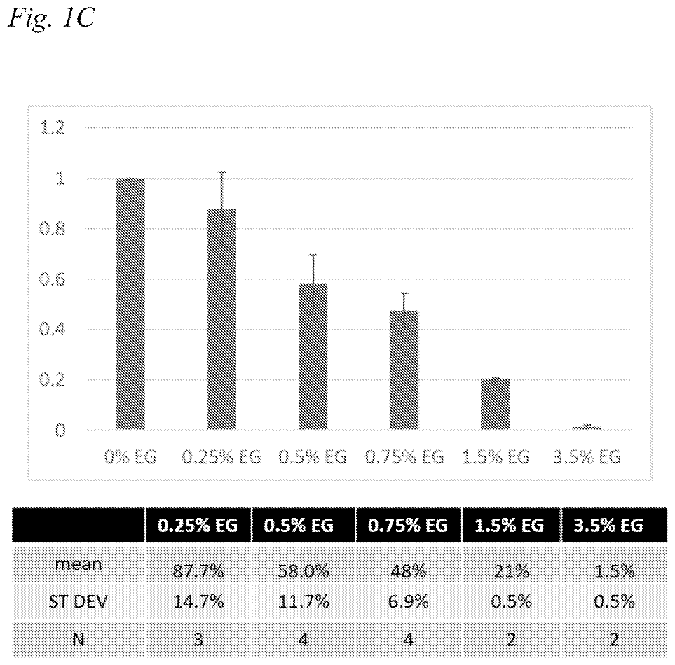

FIG. 1A depicts the results of three independent tests of SOX2-snLuc cells in the presence of varying concentrations of ethylene glycol for 48 hours. Nanoluciferase quantity normalized to tp 0 h and shown as a fraction of control cells. “TE” refers to a control population of cells exposed to differentiation media instead of pluripotency media.

FIG. 1B depicts the results of three independent tests of CGA-snLuc cells in the presence of BAP differentiation medium with varying concentrations of ethylene glycol for 65 hours. Nanoluciferase quantity is shown as a fraction of control cells. “Pluri” refers to a control population of cells exposed to pluripotency media instead of differentiation media.

The assay was also tested in the context of detecting an adulterant, ethylene glycol (EG), that is known to affect viability of mouse embryos. IC50 was calculated using the logistic 4P Hill equation, for 1-cell MEA, 2-cell MEA, MEAlt (CGA-snLuc), and MEAlt (SOX2-SnLuc) as detailed in table 2 below.

TABLE 2

| | IC50 |
|---|---|
| 1-cell MEA | 2.14% EG |
| 2-cell MEA | 3.19% EG |
| MEAlt (CGA-snLuc) | 0.77% EG |
| MEAlt (SOX2-snLuc) | 1.25% EG |

The results suggest that the iPS cells are somewhat less tolerant to ethylene glycol than are mouse embryos, although overall the sensitivity between the various systems is largely comparable. The MEAlt assays were also found to successfully detect low levels of additional adulterants, such as triton-x-100 and formaldehyde.

FIG. 1C depicts the results of multiple independent tests of CGA-snLuc cells in the presence of DAF-BAP differentiation medium with varying concentrations of ethylene glycol for 48 hours.

### EMBODIMENTS

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.

1. A method for assessing cytotoxicity of cellular media and devices, the method comprising:
exposing a first target media to a first genetically modified stem cell population, wherein the first genetically modified stem cell population comprises stem cells that have been modified to express a first reporter upon activation of a gene associated with trophectoderm development in the genetically modified stem cell population; and
detecting expression of the first reporter.
   2. The method of embodiment 1, wherein the gene associated with trophectoderm development is selected form the group comprising: CGA, and CGB.
   3. The method of any one of embodiments 1 or 2, wherein the first reporter comprises a bioluminescent reporter.
   4. The method of any one of the preceding embodiments, wherein the first genetically modified stem cell population comprises induced pluripotent stem cells that have been modified to express a first reporter upon activation of a gene associated with trophectoderm development in the genetically modified stem cell population.
   5. The method of any one of the preceding embodiments, wherein the first target media comprises a medium configured to support trophectoderm differentiation.
   6. The method of any one of the preceding embodiments, wherein the first target media comprises a control media that has been exposed to a test device.
   7. The method of any one of the preceding embodiments, wherein the first reporter is secreted into the first target media.
   8. The method of any one of the preceding embodiments, wherein the first target media comprises basal media comprising BMP-4, A83-01, and/or PD173074.
   9. The method of any one of the preceding embodiments, wherein the first target media comprises forskolin.
   10. The method of embodiment 9, wherein the first target media is exposed to the first genetically modified stem cell population for about 48 hours.
   11. The method of any one of embodiments 1 through 10, further comprising:
exposing a second target media to a second genetically modified stem cell population, wherein the second genetically modified stem cell population comprises stem cells that have been modified to express a second reporter upon activation of a gene associated with pluripotency in the genetically modified stem cells; and
analyzing the second target media for expression of the second reporter.
   12. The method for embodiment 11, wherein the gene associated with pluripotency is selected from the group comprising: SOX2, NANOG, and POUF1.
   13. The method of any one of embodiments 11 or 12, wherein the second reporter comprises a bioluminescent reporter.
   14. The method of any one of embodiments 11 through 13, wherein the second genetically modified stem cell population comprise induced pluripotent stem cells that have been modified to express a second reporter upon activation of a gene associated with pluripotency in the genetically modified stem cells.
   15. The method of any one of embodiments 11 through 14, wherein the second target media comprises a medium configured to support pluripotency.
   16. The method of any one of embodiments 11 through 15, wherein the second target media comprises a control media that has been exposed to a test device.
   17. The method of any one of the preceding embodiments, further comprising: thawing the first genetically modified stem cell population.
   18. A system for testing cell culture media, the system comprising:
a first genetically modified stem cell population, wherein the first genetically modified stem cell population comprises stem cells that have been modified to express a first reporter upon activation of a gene associated with trophectoderm development.
   19. The system of embodiment 18, wherein the gene associated with trophectoderm development is selected form the group comprising: CGA, and CGB.
   20. The system of any one of embodiments 18 or 19, wherein the first reporter comprises a bioluminescent reporter.
   21. The system of any one of embodiments 18 through 20, wherein the first genetically modified stem cell population comprises induced pluripotent stem cells.
   22. The system of any of embodiments 18 through 21, comprising:
a first target media comprising a medium configured to support trophectoderm differentiation.
   23. The system of embodiment 22, wherein the first target media comprises a control media that has been exposed to a test device.
   24. The system of any one of embodiments 22 or 23, wherein the first reporter is secreted by the stem cells.
   25. The system of any one of embodiments 22 through 24, wherein the first target media comprises basal media comprising BMP-4, A83-01, and PD173074.
   26. The system of any one of embodiments 22 through 25, wherein the first target media comprises forskolin.
   27. The system of any one of embodiments 18 through 26, further comprising:
a second genetically modified stem cell population, wherein the second genetically modified stem cell population comprises stem cells that have been modified to express a second reporter upon activation of a gene associated with pluripotency.
   28. The system for embodiment 27, wherein the gene associated with pluripotency is selected from the group comprising: SOX2, NANOG, and POUF1.
   29. The system of any one of embodiments 27 or 28, wherein the second reporter comprises a bioluminescent reporter.
   30. The system of any one of embodiments 27 through 29, wherein the second genetically modified stem cells comprise induced pluripotent stem cells.
   31. The system of any of embodiments 27 through 30, comprising:
a second target media comprising a medium configured to support pluripotency.
   32. The system of any embodiment 31, wherein the second target media comprises a control media that has been exposed to a test device.
   33. A cell culture comprising:
a first genetically modified stem cell population, wherein the first genetically modified stem cell population comprises stem cells that have been modified to express a first reporter upon activation of a gene associated with trophectoderm development.
   34. The cell culture of embodiment 33, wherein the gene associated with trophectoderm development is selected form the group comprising: CGA, and CGB.
   35. The cell culture of any one of embodiments 33 or 34, wherein the first reporter comprises a bioluminescent reporter.
   36. The cell culture of any one of embodiments 33 through 35, wherein the first genetically modified stem cell population comprises induced pluripotent stem cells.
   37. The cell culture of any one of embodiments 33 through 36, wherein the first genetically modified stem cell population is frozen.
   38. A laboratory testing kit comprising:
a first genetically modified stem cell population, wherein the first genetically modified stem cell population comprises stem cells that have been modified to express a first reporter upon activation of a gene associated with trophectoderm development, wherein said first genetically modified stem cell population is contained within a sterile package.
   39. The laboratory testing kit of embodiment 38, wherein the gene associated with trophectoderm development is selected from the group comprising: CGA, and CGB.
   40. The laboratory testing kit of any one of embodiments 38 or 39, wherein the first reporter comprises a bioluminescent reporter.
   41. The laboratory testing kit of any one of embodiments 38 through 40, wherein the first genetically modified stem cell population comprises induced pluripotent stem cells that have been modified to express a first reporter upon activation of a gene associated with trophectoderm development.
   42. The laboratory testing kit of any of embodiments 38 through 41, comprising:
a first target media comprising a medium configured to support trophectoderm differentiation.
   43. The laboratory testing kit of embodiment 42, wherein the first target media comprises forskolin.
   44. The laboratory testing kit of any one of embodiments 38 through 43, further comprising:
a second genetically modified stem cell population, wherein the second genetically modified stem cell population comprises stem cells that have been modified to express a second reporter upon activation of a gene associated with pluripotency, wherein said second genetically modified stem cell population is contained within a sterile package.
   45. The laboratory testing kit for embodiment 44, wherein the gene associated with pluripotency is selected from the group comprising: SOX2, NANOG, and POUF1.
   46. The laboratory testing kit of any one of embodiments 44 or 45, wherein the second reporter comprises a bioluminescent reporter.
   47. The laboratory testing kit of any one of embodiments 44 through 46, wherein the second genetically modified stem cells comprise induced pluripotent stem cells that have been modified to express a second reporter upon activation of a gene associated with pluripotency.
   48. The laboratory testing kit of any of embodiments 44 through 47, comprising:
a second target media comprising a medium configured to support pluripotency.

The use of the terms “a” and “an” and “the” and similar referents in the context of describing the invention especially in the context of the following claims are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method for assessing cytotoxicity of cellular media and devices, the method comprising:
exposing a first target media to a first genetically modified stem cell population, wherein the first genetically modified stem cell population comprises stem cells that have been modified to express a first reporter upon activation of a gene associated with trophectoderm development in the genetically modified stem cell population; and
detecting expression of the first reporter.

2. The method of claim 1, wherein the gene associated with trophectoderm development is selected form the group comprising: CGA, and CGB.

3. The method of claim 1, wherein the first reporter comprises a bioluminescent reporter.

4. The method of claim 1, wherein the first genetically modified stem cell population comprises induced pluripotent stem cell.

5. The method of claim 1, wherein the first target media comprises a medium configured to support trophectoderm differentiation, and/or wherein the first target media comprises a control media that has been exposed to a test device.

6. (canceled)

7. The method of claim 1, wherein the first reporter is secreted into the first target media.

8. The method of claim 1, wherein the first target media comprises basal media comprising BMP-4, A83-01, and/or PD173074, and/or wherein the first target media comprises forskolin.

9. (canceled)

10. The method of claim 8, wherein the first target media comprises forskolin and wherein the first target media is exposed to the first genetically modified stem cell population for about 48 hours.

11. The method of claim 1, further comprising:
exposing a second target media to a second genetically modified stem cell population, wherein the second genetically modified stem cell population comprises stem cells that have been modified to express a second reporter upon activation of a gene associated with pluripotency in the second genetically modified stem cell population, and optionally wherein the second genetically modified stem cells comprise induced pluripotent stem cells; and
detecting expression of the second reporter.

12. The method for claim 11, wherein the gene associated with pluripotency is selected from the group comprising: SOX2, NANOG, and POUF1.

13. The method of claim 11, wherein the second reporter comprises a bioluminescent reporter.

14. (canceled)

15. The method of claim 11, wherein the second target media comprises a medium configured to support pluripotency, and/or wherein the second target media comprises a control media that has been exposed to a test device.

16. (canceled)

17. (canceled)

18. A system for testing cell culture media, the system comprising:
a first genetically modified stem cell population, wherein the first genetically modified stem cell population comprises stem cells that have been modified to express a first reporter upon activation of a gene associated with trophectoderm development.

19. The system of claim 18, wherein the gene associated with trophectoderm development is selected form the group comprising: CGA, and CGB.

20. The system of claim 18, wherein the first reporter comprises a bioluminescent reporter, and/or wherein the first reporter is secreted by the stem cells.

21. (canceled)

22. The system of claim 18, comprising:
a first target media comprising a medium configured to support trophectoderm differentiation, and/or wherein the first target media comprises a control media that has been exposed to a test device.

23. (canceled)

24. (canceled)

25. The system of claim 22, wherein the first target media comprises basal media comprising BMP-4, A83-01, and PD173074, and/or wherein the first target media comprises forskolin.

26. (canceled)

27. The system of claim 18, further comprising:
a second genetically modified stem cell population, wherein the second genetically modified stem cell population comprises stem cells that have been modified to express a second reporter upon activation of a gene associated with pluripotency.

28. The system for claim 27, wherein the gene associated with pluripotency is selected from the group comprising: SOX2, NANOG, and POUF1.

29. The system of claim 27, wherein the second reporter comprises a bioluminescent reporter.

30. (canceled)

31. The system of claim 27, comprising:
a second target media comprising a medium configured to support pluripotency, and/or wherein the second target media comprises a control media that has been exposed to a test device.

32. (canceled)

33. A cell culture comprising:
a first genetically modified stem cell population, wherein the first genetically modified stem cell population comprises stem cells that have been modified to express a first reporter upon activation of a gene associated with trophectoderm development.

34. The cell culture of claim 33, wherein the gene associated with trophectoderm development is selected form the group comprising: CGA, and CGB.

35. The cell culture of claim 33, wherein the first reporter comprises a bioluminescent reporter.

36. The cell culture of claim 33, wherein the first genetically modified stem cell population comprises induced pluripotent stem cells.

37. (canceled)

38. A laboratory testing kit comprising:
a first genetically modified stem cell population, wherein the first genetically modified stem cell population comprises stem cells that have been modified to express a first reporter upon activation of a gene associated with trophectoderm development, wherein said first genetically modified stem cell population is contained within a sterile package.

39. The laboratory testing kit of claim 38, wherein the gene associated with trophectoderm development is selected from the group comprising: CGA, and CGB.

40. The laboratory testing kit of claim 38, wherein the first reporter comprises a bioluminescent reporter.

41. The laboratory testing kit of claim 38, wherein the first genetically modified stem cell population comprises induced pluripotent stem cells.

42. The laboratory testing kit of claim 38, comprising:
a first target media comprising a medium configured to support trophectoderm differentiation, and optionally wherein the first target media comprises forskolin.

43. (canceled)

44. The laboratory testing kit of claim 38, further comprising:
a second genetically modified stem cell population, wherein the second genetically modified stem cell population comprises stem cells that have been modified to express a second reporter upon activation of a gene associated with pluripotency, wherein said second genetically modified stem cell population is contained within a sterile package, and optionally wherein the second genetically modified stem cells comprise induced pluripotent stem cells.

45. The laboratory testing kit for claim 44, wherein the gene associated with pluripotency is selected from the group comprising: SOX2, NANOG, and POUF1.

46. The laboratory testing kit of claim 44, wherein the second reporter comprises a bioluminescent reporter.

47. (canceled)

48. The laboratory testing kit of claim 44, comprising:
a second target media comprising a medium configured to support pluripotency.
